(19) United States
(12) Patent Application Publication
(10) Pub. No.: US 2017/0182195 A1
(43) Pub. Date: Jun. 29, 2017

(54) VENT VALVE ARRANGEMENT FOR A STERILIZATION CONTAINER HELD UNDER VACUUM

(71) Applicant: Peter WAGNER, Starnberg (DE)

(72) Inventor: Peter WAGNER, Starnberg (DE)

(21) Appl. No.: 15/034,325

(22) PCT Filed: Nov. 4, 2014
(86) PCT No.: PCT/EP2014/002947

§ 371 Date: Oct. 19, 2016

(30) Foreign Application Priority Data
Nov. 4, 2013 (DE) 10 2013 018 499.1

(51) Int. Cl. A61L 2/26; A61B 50/30; F16K 24/06; A61L 2/07

(52) U.S. Cl. CPC A61L 2/26; A61L 2/07; A61B 50/30; F16K 24/06; A61L 2202/14

(57) ABSTRACT

A vent valve arrangement for a sterilization container held under vacuum after completion of the sterilization, which container must be depressurized for opening, comprising a vent valve capsule which comprises a cavity enclosed by a circumferential wall, which cavity is closed on a first end by a manually irreversibly detachable cover and comprises an opening on the second end thereof. The capsule is designed such that prior to the sterilization of the sterilization container, it can be inserted under external seal into an opening of a wall of the sterilization container or a component thereof, in such a way that the end of the cavity closed by the cover lies on the outer side of the sterilization container and closes this opening in a gas-tight manner, while the cavity over the opening is constantly in connection to the interior of the sterilization container.

The invention relates to a vent valve arrangement for a sterilisation container held under vacuum after completion of the sterilisation, of the kind cited in the preamble of claim 1.

Sterilisation containers or sterile containers are reusable sterile goods packagings. They are used for holding surgical instruments or surgical textiles or other items to be sterilised (implants, machine parts etc.) during sterilisation as well as for maintaining the sterility of the sterilised content during transportation or subsequent storage.

Such sterilisation containers, on the one hand, shall be as permeable as possible during a sterilisation procedure in order not to obstruct sterilisation of the content, but on the other hand, shall be as impermeable/tight as possible after completed sterilisation in order to protect the content in the best possible way against recontamination.

Sterilisation containers are known, which have valve arrangements or surface filters or labyrinth-like bypass-screens based on the “Pasteur” principle arranged on them, and which are designed in such a way that their permeability is high enough so as not to hinder sterilisation, whilst their impermeability/tightness against particle penetration is just about sufficient to prevent the entry of microorganisms into the sterilisation container after sterilisation. Designing such filters so that they meet all respective operating and safety requirements is therefore a difficult task.

Furthermore the U.S. Pat. No. 4,196,166 A and the U.S. Pat. No. 5,352,416 A as well as the EP 1169 068 B1 have disclosed sterilisation containers of the kind mentioned in the beginning, which by means of suitable controls or valve arrangements during sterilisation within a steriliser can be held in an open and permeable state, which facilitates completely unhindered sterilisation, until this sterilisation container is closed at a predetermined point in time. The valve arrangements may e.g. be arranged in a lid or bottom part of the sterilisation container or even in other places thereof. If e.g. this closing is effected within the steriliser at a point in time during the sterilisation procedure, at which the sterilisation container just happens to be under vacuum, this vacuum can be maintained by closing the sterilisation container such that the content of the sterilisation container is then vacuum-packed.

Especially following a sterilisation with saturated steam, such a vacuum can be built up or such an already existing vacuum can be further increased by subsequently cooling the sterile but still very warm air present in the already closed sterilisation container, and during this time, because due to the gas-tight sealing no gas can intrude from the outside, a vacuum is created or an already existing partial vacuum is further increased inside thiss sterilisation container.

If such a vacuum which was preserved by the sterilisation process continues to exist after all handling and storage, the sterilisation container in question was/is obviously gas-tight, and therefore must evidently also have formed an effective barrier against microorganisms. Such vacuum-sealed sterilisation containers therefore can, for each individual application via the enclosed vacuum, furnish proof of physical integrity of the strerilising container and proof that the sterile barrier has indeed been maintained intact until the moment of opening and no microorganisms could have penetrated.

To open a vacuum-sealed sterilisation container, the vacuum must however first be relieved or discharged so that the lid which is pressed onto the lower bottom part by the differential pressure can be removed. This opening can be effected as intended at the place of usage, I.e. in a very clean operating theatre with germ-free ambient air by operating a vent valve, through which, at the moment of operating this valve, air can flow into the sterilisation container, thus relieving the vacuum maintained up to this point in time.

For additional safety a permanent filter or particle separator may be arranged below such a vent valve in the flow profile, the sole purpose of which consists in freeing the few litres of inflowing air required for pressure equalisation from any particles introduced during the process of opening, in order to prevent the content of the sterilisation container from being contaminated up to the point in time of opening.

However, known vent valves with or without downstream filters have considerable disadvantages: they are part of the sterilisation container and must be disinfected by washing and subsequently sterilised as must any other part of the container, with each new use cycle, which in particular considerably restricts the choice of materials which can be used for the filter or particle separator.

In addition, with such vent valves, there exists the even unlikely risk that the vent valve is handled or manipulated incorrectly, e.g. in such a way as to result in an only partial venting of the sterilisation container, wherein a possibly still existing residual vacuum after such manipulation would falsely continue to indicate integrity.

Furthermore, especially for the sterilisation with saturated steam, moisture may condense in a vacuum-sealed sterilisation container during the described cooling because the warm air enclosed in the sterilisation container may possibly contain more moisture dissolved in gaseous form than it can hold in a cold state. During cooling this may then possibly lead to reaching the dew point, which becomes noticeable in the form of condensation preferably on the walls of the sterilisation container. This is sterile moisture which is not dangerous from a hygienic point of view, but users of “constantly permeable” filter containers, where such moisture is simply passed “in a hidden manner” to the dry ambient air during storage due to the existing permanent exchange with the direct environment of the sterilisation container, are used to dry results. For this reason a hydrophilic material such as a silica gel/silica gel granulate is normally used in the case of a vacuum-sealed sterilisation container, which in the closed sterilisation container can create a relative air humidity of <40%, which however necessitates additional expense.

The invention is based on the requirement to propose an improved vent valve arrangement of the kind mentioned in the beginning, which is compact and intrinsically safe to manipulate and which does not have to be subjected to disinfection by washing.

This requirement is met by the characteristics mentioned in claim 1.

Advantageous designs and further developments of the invention are revealed in the sub-claims.

The vent valve arrangement according to the invention is destined for a single venting of a sterilisation container preserving a vacuum after sterilisation during handling and storage.

For this purpose the vent valve arrangement is fitted or pressed into a suitable additional opening of the sterilisation container or of one of its components from the outside prior to the intended sterilisation, and in this inserted state seals this opening in a gas-tight manner. The vent valve arrangement, in this inserted state, can no longer be removed from this opening without being damaged; only once the sterilisation container has been opened, can the vent valve arrangement be pressed out of the opening again from inside to outside in the direction contrary to the insertion direction.

The vent valve arrangement comprises a cavity, which is in contact with the sealed interior of the sterilisation container, when the vent valve arrangement is inserted into the sterilisation container.

This cavity preferably contains a filter or particle separator and/or a drying agent, which because the cavity of the pressed-in vent valve arrangement is in constant contact with the interior of the sterilisation container is just as permanently accessible for the sterilisation medium during sterilisation as the interior of the sterilisation container itself, and which therefore is also automatically sterilised at the same time.

Due to the single use of the vent valve arrangement, the filter or particle separator placed in the cavity does not have to be designed to also withstand chemical disinfection by washing. This permits a very effective design of the component from almost any materials and could even be designed in the form of a much more effective depth filter rather than as a surface filter with a comparatively high flow resistance.

With the design including a drying agent, the vent valve arrangement can also regulate the air humidity in the sealed gas chamber of a sterilisation container preserving a vacuum, wherein the sterilisation container can be vented through a filter/particle separator preferably additionally contained in the vent valve arrangement, when the seal is actuated or deliberately destroyed, right through the destroyed seal.

Since the venting air with this design passes, of necessity, through the cavity of the inserted component containing a filter or a particle separator and/or a drying agent, it is ensured that the venting air required for relieving the vacuum within the sterilising container is always admitted through a fresh and even sterilised filter and is therefore sure to be germ-free and also dry.

The sealing of the vent valve arrangement and thus of the sterilisation container towards the outside is preferably accomplished by a covering of the top of the vent valve arrangement, which is gas-tight and can be easily opened and destroyed, so that there is no need for an additional safety seal which should indicate whether the vent valve has been actuated.

Since this top covering is arched towards the inside in case of an existing vacuum in the sterilisation container, it can even indicate whether the vent valve is intact and whether there still exists a vacuum inside the container.

The design of the vent valve arrangement according to the invention thus forms a very compact arrangement destined for single use, and which at the same time represents a vent valve, a seal, a filter or particle separator and a drying aid.

The invention will now be explained in more detail with reference to embodiments depicted in the drawing, in which

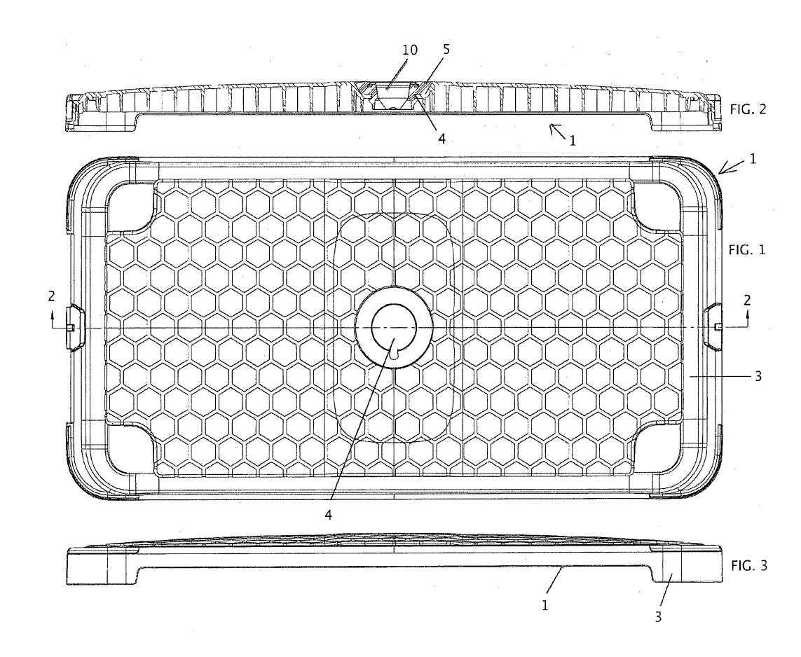

FIG. 1 shows a bottom view of a lid of a sterilisation container, where an embodiment of the vent valve arrangement is used;

FIG. 2 shows a sectional view of the lid of FIG. 1 along line 2-2 in FIG. 1;

FIG. 3 shows a lateral view of the lid depicted in FIGS. 1 and 2;

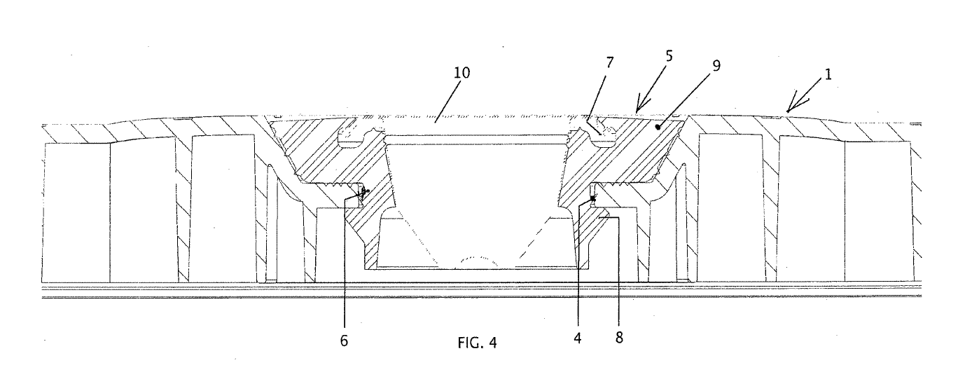

FIG. 4 shows an enlarged partial view of FIG. 2, showing a seal of the vent valve arrangement in a recess of the lid;

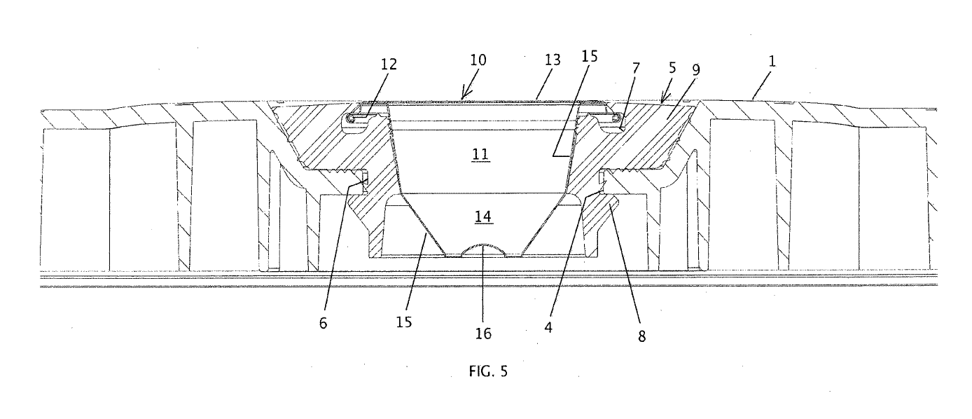

FIG. 5 shows a partial view of FIG. 2, showing the arrangement of the vent valve arrangement in the seal inserted into the lid;

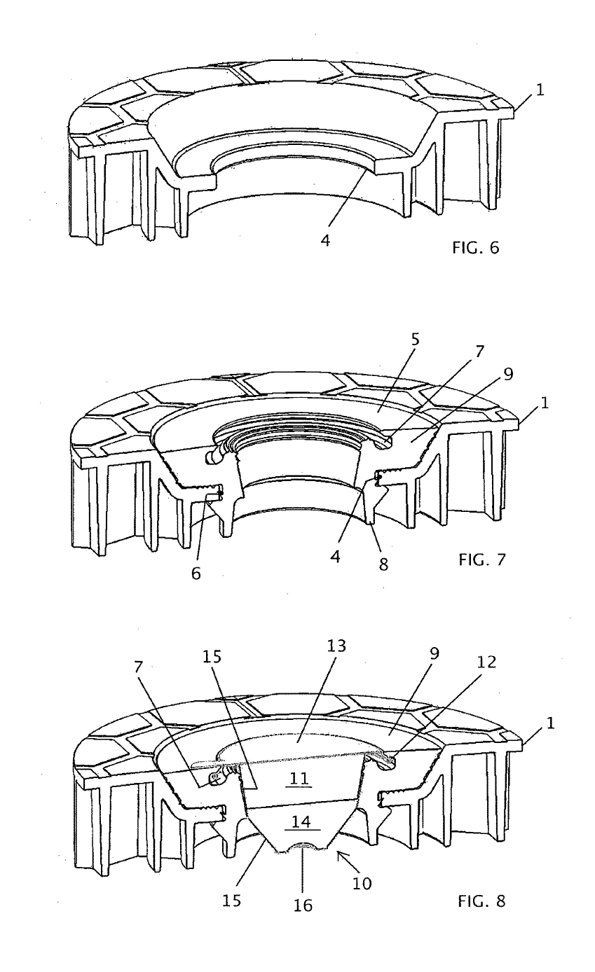

FIG. 6 shows an embodiment of the recess for receiving the vent valve arrangement in the lid,

FIG. 7 shows the embodiment according to FIG. 6 with the seal inserted;

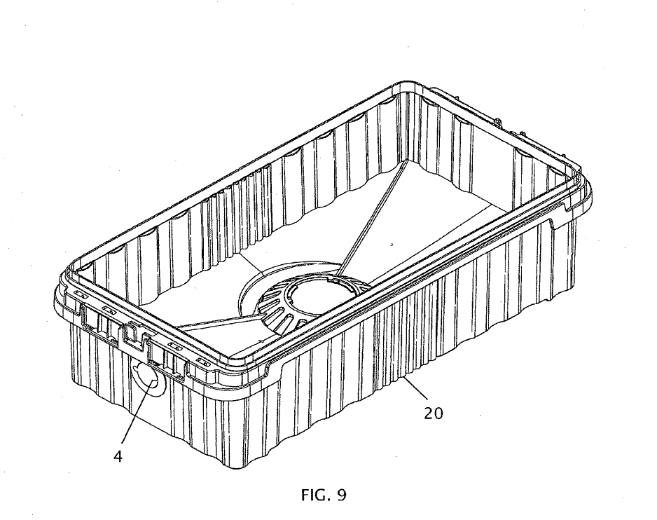

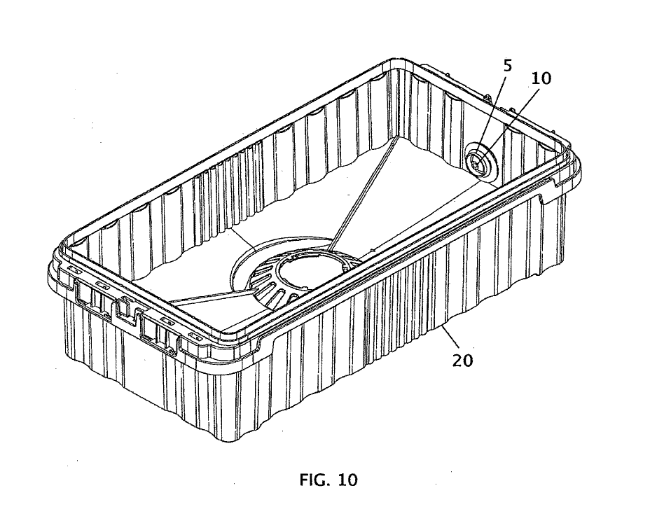

FIG. 8 shows the embodiment shown in FIGS. 6 and 7 with a vent valve capsule inserted into the seal;

FIG. 9 shows an embodiment of the lower part of the sterilisation container, showing a further possibility for placing the vent valve arrangement.

In FIGS. 1 to 3 an embodiment of a lid 1 of a sterilisation container is shown, which further comprises a trough-shaped lower part, the general shape of which is shown e.g. in FIG. 9.

FIG. 1 shows a bottom view of the lid, while FIG. 2 shows a sectional view of the lid 1 according to FIG. 1 along line 2-2.

The lid 1 has an opening 4, into which an embodiment of the vent valve arrangement according to the invention can be inserted, which comprises a vent valve capsule 10 closed by a covering 13 on its outside and a seal 5.

The vent valve capsule 10 is sealingly inserted into the opening 4.

The opening 4 is preferably arranged on the bottom of a recess in the top of lid 2 so that the outwardly protruding parts of the vent valve arrangement are arranged in the lid 1 in a protected manner.

This design of the opening or the vent valve arrangement is shown on an enlarged scale in FIGS. 4 and 5, wherein it can further be seen from FIGS. 6 to 8 that the seal 5 is initially fitted into the opening 5 (FIGS. 4 and 7), which seal comprises a circumferential groove 6, which is formed on an internal flange in the opening 4 in order to ensure a secure fit of the seal 5, wherein this circumferential groove 6 is formed between an inner part 8 and an outer part 9 of the seal.

The seal 5 further comprises an inner annular groove 7, into which a circumferential flange 12 of the vent valve capsule 10 still to be described in more detail further below, is to be fitted, the capsule being only schematically indicated in FIGS. 4 and 7. This vent valve capsule 10 comprises a cavity visible in particular in FIGS. 5 and 8, which is closed by the covering 13 attached to the circumferential flange 12. Prior to closing the cavity with the covering 13, a drying agent 14 and above it a filter or a particle separator (not shown) is preferably inserted into the cavity. The thus filled and sealed capsule can be opened only from outside by destroying the covering and is intended for single use only.

The drying agent may, for example, be silica gel, zeolite, bentonite or Montmorillonite.

Because there is now no longer any need for chemical disinfection, the filter or particle separator may be designed in a highly efficient manner. Specifically, the filter need not, as is common, be configured according to the principle of a membrane filter or surface filter, but may be designed according to the principle of a very effective depth filter because the cavity is of considerable length, which may be used as constructional space.

Precisely due to this available constructional space it is also possible that the drying agent, specifically when used in granular form, may simultaneously form the filter.

The underside of the capsule 10 which lies inside the sterilisation container, is in contact via an opening 16 with the interior of the sterilisation container so that the cavity and the parts therein can also be exposed during sterilisation to the sterilisation medium.

Starting from the outside of the opening 4, the vent valve capsule 10 with its outer flange 12 is pressed into the annular groove 7 of the seal 5, wherein the vent valve capsule is covered on its top by the covering 13, which albeit can be easily destroyed by perforating or tearing it open, but which preferably, without destroying it, is optically visibly deformable when subjecting it to a uniform load. As such, if a vacuum exists inside the sterilisation container, the covering may arch inwards and indicate, through this optically visible arching, as to whether a vacuum exists in the sterilisation container or not.

When the sterilisation container is to be opened, the covering 13 is pulled off the circumferential flange 12 or destroyed in some other way, allowing air to flow through the cavity and the functional elements arranged in there into the sterilisation container, in order to permit removal of the lid 1 from the lower part of the sterilisation container.

The air flowing into the sterilisation container when the covering 13 is opened, is of necessity passed through the cavity of the vent valve, wherein the means arranged therein filter out or separate any particles contained in the air current, and wherein the optionally existing drying agent even dries this air. If the cavity is, as is preferable, equipped with a highly efficient particle filter configured as a depth filter with low flow resistance, then this will ensure, even during quick venting, that no microorganisms even as small as viruses, can enter into the sterilisation container.

FIG. 9 shows an embodiment of a sterilisation container, where the vent valve arrangement is arranged, not in the lid, but in a side wall of the lower part 20 of the sterilisation container.

The arrangement of the vent valve arrangement in the lid or in a side wall of the sterilisation container represents only two possible embodiments. The capsule of the vent valve arrangement may equally be fitted to other components of a sterilisation container, such as to other valve arrangements, and the capsule itself, for a corresponding design of the opening 4 in the sterilisation container or the respective components, may be provided with its own seal again destined for single use only, so that both the capsule and the associated seal can be fitted as a unit into an opening of the sterilisation container, without changing the features of the capsule in other respects.

Furthermore it is of course possible for the capsule to be given another outer shape, as long as it can be fitted into an opening of the sterilisation container or components thereof, either sealed via a seal or provided with its own sealing elements.

1. A vent valve arrangement for a sterilisation container held under vacuum after completion of the sterilisation, which container must be depressurised for opening, characterised in that the vent valve arrangement comprises a vent valve capsule (10) which comprises a cavity enclosed by a circumferential wall (15), which cavity is sealed on a first end by a manually irreversibly detachable cover (13) and comprises an opening (16) on the second end thereof, and in that the capsule (10) is designed such that prior to the sterilisation of the sterilisation container it can be inserted under external seal into an opening (4) of a wall of the sterilisation container or a component thereof, in such a way that the end of the cavity closed by the cover (13) lies on the outer side of the sterilisation container and closes this opening (4) in a gas-tight manner, while the cavity, via the opening (16), is constantly in connection with the interior of the sterilisation container.

2. The vent valve arrangement according to claim 1, characterised in that the circumferential wall (15) of the capsule (10) comprises, at the end adjacent to the covering, a circumferential flange (12), to which the covering (13) is attached.

3. The vent valve arrangement according to claim 2, characterised in that the circumferential flange (12) is arranged in a recess of the wall of the sterilisation container or one of its components such that a non-damaging removal of the capsule (10) inserted from outside into the sterilisation container is not possible until the sterilisation container has been opened.

4. The vent valve arrangement according to one of claims to 3, characterised in that opening, damaging or destroying the covering (13) is effected irreversibly by e.g. tearing it open, pressing it in or perforating it.

5. The vent valve arrangement according to one of claims 1 to 4, characterised in that the cavity contains a sterilisable filter (11) or a particle separator.

6. The vent valve arrangement according to claim 5, characterised in that the filter (11) is a germ-free filtering surface filter or membrane filter.

7. The vent valve arrangement according to claim 5, characterised in that the filter (11) is a highly efficient particle filter according to the principle of a depth filter.

8. The vent valve arrangement according to claim 5, characterised in that the particle separator is designed according to the principle of a “Pasteur” path.

9. The vent valve arrangement according to one of the preceding claims, characterised in that the cavity includes a sterilisable drying agent.

10. The vent valve arrangement according to claim 9, characterised in that the drying agent is silica gel, zeolite, bentonite or montmorillonite.

11. The vent valve arrangement according to one of the preceding claims, characterised in that the drying agent is also the filter.

12. The vent valve arrangement according to one of claims 9 to 11, characterised in that the drying agent, which is in contact with the interior of the sterilisation container is designed such that during storage a relative air humidity of <40% can form inside the gas-tight sealed sterilisation container.

13. The vent valve arrangement according to one of the preceding claims, characterised in that the absence of a vacuum can be detected by the absence of an inwardly directed arching or by damage or destruction of the top of the covering (13).

14. The vent valve arrangement according to one of the preceding claims, characterised in that the cavity of the capsule (10) contains a passive computer-readable chip.

15. The vent valve arrangement according to one of the preceding claims, characterised in that the computer-readable chip is a programmable RFID chip.

16. The vent valve arrangement according to claim 14 or 15, characterised in that the passive electronic chip is configured to sample the vacuum of the sterilisation container.
